(19) United States
(12) Patent Application Publication
(10) Pub. No.: US 2019/0102881 A1
(43) Pub. Date: Apr. 4, 2019

(54) ENDOSCOPE APPARATUS AND CAMERA CONTROL UNIT

(71) Applicant: OLYMPUS CORPORATION, Tokyo (JP)

(72) Inventors: Takehide FUJIMOTO, Tokyo (JP); Nanako UBAYAMA, Tokyo (JP); Hitoshi KOMINE, Tokyo (JP)

(73) Assignee: OLYMPUS CORPORATION, Tokyo (JP)

(21) Appl. No.: 16/154,828

(22) Filed: Oct. 9, 2018

Related U.S. Application Data
(63) Continuation of application No. PCT/JP2017/010896, filed on Mar. 17, 2017.

(30) Foreign Application Priority Data
Apr. 28, 2016 (JP) 2016-091341

(51) Int. Cl. G06T 7/00; G02B 23/24; H04N 5/217; H04N 5/225; G06T 5/20; A61B 1/00

(52) U.S. Cl. CPC G06T 7/0012; G02B 23/2476; H04N 5/2173; G06T 2207/10068; G06T 5/20; A61B 1/00043; H04N 5/2254

(57) ABSTRACT

An endoscope apparatus includes: a first discrimination section configured to be connectable to a first endoscope including a communication circuit and capable of transmitting image pickup device type information, and a second endoscope including a resistance configured by a resistance value for enabling identification of a mounted image pickup device and corresponding to the image pickup device, and configured to acquire information for identifying a type of an image pickup device from a connected endoscope to determine a type of the connected endoscope; and an image pickup communication reception section configured to receive the image pickup device type information from the communication circuit when it is determined in the first discrimination section that the first endoscope is connected.

### CROSS REFERENCE TO RELATED APPLICATION

This application is a continuation application of PCT/JP2017/010896 filed on Mar. 17, 2017 and claims benefit of Japanese Application No. 2016-091341 filed in Japan on Apr. 28, 2016, the entire contents of which are incorporated herein by this reference.

### BACKGROUND OF THE INVENTION

### Field of the Invention

The present invention relates to an endoscope apparatus and a camera control unit to which a plurality of endoscopes with different type determination methods can be connected.

### Description of Related Art

In recent years, endoscope apparatuses have been used in various fields such as the medical field and the industrial field. In the medical field, an endoscope apparatus is used for observation of organs in a body cavity, therapeutic treatment using treatment instruments, surgical operation under observation with an endoscope, and the like. As an endoscope apparatus, an electronic endoscope is often adopted which is configured to be able to pick up a picked-up image within a patient's body cavity with an image pickup device. The endoscope apparatus has a camera control unit configured to perform video processing on a picked-up image obtained by picking up an image through the electronic endoscope, and the camera control unit can convert the picked-up image into a video signal for output on a monitor or recording, for example.

The endoscope is configured to be removably connected to the camera control unit via a cable. The image pickup device provided in the endoscope is configured to supply the picked-up image to the camera control unit via the cable, and also to receive power supply from the camera control unit. Various kinds of endoscopes can be connected to the camera control unit. Various kinds of image pickup devices can be adopted as an image pickup device built into the endoscope. The camera control unit is configured to detect a type of the image pickup device mounted on the endoscope, and to perform optimal driving according to the type of the image pickup device.

In order to determine the type of the image pickup device mounted on the endoscope, a resistive voltage dividing scheme may be adopted. The resistive voltage dividing scheme is for providing the endoscope with a resistance having a resistance value corresponding to the type of the image pickup device, and calculating a resistive voltage dividing value due to the resistance to determine the type of the image pickup device.

However, in a commonly used type determination method for an image pickup device based on resistive voltage dividing, it is necessary to set a margin for a voltage value for determining each type, so it is difficult to discriminate a vast number of image pickup devices. And the resistive voltage dividing scheme may erroneously detect the type of an image pickup device due to contact resistance, corrosion of connector pins, or the like. So, a communication scheme may be adopted which is for providing the endoscope with a communication circuit and exchanging information between the communication circuit of the endoscope and a communication circuit of the camera control unit to determine the type of the image pickup device.

Japanese Patent Application Laid-Open Publication No. 2006-055350 discloses an endoscope apparatus which adopts such a communication scheme to receive a scope ID including image pickup device information and detect a scope type to perform power supply control and clock control.

Note that an endoscope connectable to the camera control unit does not necessarily adopt the communication scheme, but may adopt the resistive voltage dividing scheme.

### SUMMARY OF THE INVENTION

An endoscope apparatus according to an aspect of the present invention includes: a first discrimination section configured to be connectable to a first endoscope including a communication circuit and capable of transmitting image pickup device type information, and a second endoscope including a resistance configured by a resistance value for enabling identification of a mounted image pickup device and corresponding to the image pickup device, and configured to acquire information for identifying a type of an image pickup device from a connected endoscope to identify a type of the connected endoscope; and an image pickup communication reception section configured to receive the image pickup device type information from the communication circuit when it is determined in the first discrimination section that the first endoscope is connected.

A camera control unit according to an aspect of the present invention is a camera control unit for driving an image pickup device mounted on an endoscope, including: a connection section connectable to a first endoscope including a storage area in which image pickup device type information enabling identification of a mounted image pickup device is stored, and to a second endoscope including a resistance configured by a resistance value for enabling identification of a mounted image pickup device and corresponding to the image pickup device; a circuit configured to acquire a signal for identifying a type of the image pickup device mounted on the second endoscope through the connection section when the first endoscope or the second endoscope is connected to the connection section; and a first discrimination section configured to determine whether an endoscope connected to the connection section is the first endoscope or the second endoscope based on the signal acquired by the circuit.

### BRIEF DESCRIPTION OF THE DRAWINGS

FIG. 1 is a block diagram showing an endoscope apparatus according to an embodiment of the present invention;

FIG. 2 is a flowchart showing processes in an analog front end section 20;

FIG. 3 is a flowchart showing processes in a video processing section 30;

FIG. 4A is a flowchart showing processes in an endoscope 40;

FIG. 4B is a flowchart showing processes in the endoscope 40; and

FIG. 5 is a timing chart for explaining timing of CLK output, image pickup device communication, and scope communication.

### DETAILED DESCRIPTION OF PREFERRED EMBODIMENT

An embodiment of the present invention will be described below in detail with reference to the drawings.

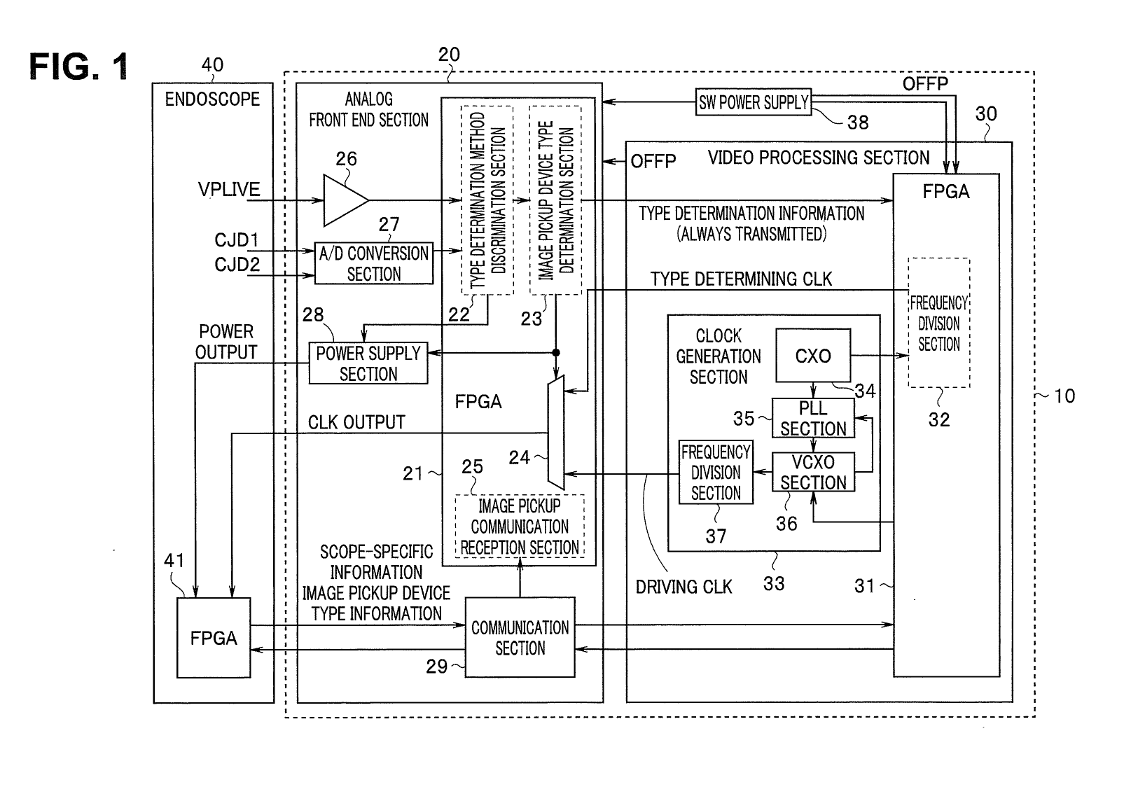

FIG. 1 is a block diagram showing an endoscope apparatus according to an embodiment of the present invention. Although the endoscope apparatus in FIG. 1 is composed of a camera control unit 10 and an endoscope 40, not only the endoscope 40 but also another endoscope not shown can be removably connected to the camera control unit (hereinafter also referred to as CCU) 10. The endoscope 40 may adopt any of the resistive voltage dividing scheme and the communication scheme as a type discriminating scheme for an image pickup device, and although for simplicity of explanation FIG. 1 shows an example in which the endoscope 40 supports these two schemes, the endoscope 40 actually supports only either one of the schemes. Whether the endoscope adopts the resistive voltage dividing scheme or the communication scheme as the type determining scheme for the image pickup device, the endoscope apparatus in the embodiment efficiently performs hybrid type determination supporting both schemes. Note that regarding the CCU 10 and the endoscope 40, FIG. 1 shows only components for type determination of the image pickup device, and illustration of a circuit for image pickup and a circuit for image processing is omitted.

In an endoscope adopting the communication scheme as the type determining scheme for the image pickup device, first of all, in order to determine the type, it is necessary to supply a communication circuit with power and a clock. On the other hand, in an endoscope adopting the resistive voltage dividing scheme as the type determining scheme for the image pickup device, in order to start up normally, it may be necessary to supply the endoscope with powers having a plurality of voltages in a sequence corresponding to the type of the image pickup device or the type of an element in the endoscope. Accordingly, when an endoscope adopting the resistive voltage dividing scheme is supplied with power and a clock for type determination in the communication scheme, the endoscope may not start normally. So, the embodiment is configured to perform type determination based on the resistive voltage dividing scheme before type determination based on the communication scheme. Note that an endoscope which will be adopted in the next generation will often adopt the communication scheme, and when type determination based on the resistive voltage dividing scheme does not work, it may be considered that a next-generation endoscope is connected to the CCU 10.

The CCU 10 is composed of an analog front end section 20 and a video processing section 30. A switching (SW) power supply 38 generates various powers to be used in the CCU 10. Power output from the SW power supply 38 is also supplied to an FPGA (field programmable gate array) 31 of the video processing section 30 and to an FPGA 21 of the analog front end section 20. Note that when power output from the SW power supply 38 is supplied to the video processing section 30, an OFFP signal indicating power-on is supplied to the video processing section 30. And the video processing section 30 is configured to supply the OFFP signal to the FPGA 21 of the analog front end section 20.

The analog front end section 20 has the FPGA 21 for type determination of the image pickup device. The analog front end section 20 has an amplifier 26 and an A/D conversion section 27 in order to enable type determination of the image pickup device based on the resistive voltage dividing scheme. The analog front end section 20 has a power supply section 28 and a communication section 29 in order to enable type determination of the image pickup device based on the communication scheme. Note that as the communication section 29, for example, a CPLD (complex programmable logic device) can be adopted. The video processing section 30 is provided with the FPGA 31 and a clock generation section 33.

For the endoscope 40, an image pickup device, not shown, such as a CCD or CMOS sensor is disposed at a tip of an insertion portion not shown at a tip of the endoscope 40, for example. The endoscope 40 is continuously provided with an operation portion at a base end side of the insertion portion, and is extendingly provided with a cable not shown from the operation portion. The endoscope 40 is configured to be removably connected to the camera control unit 10 through an endoscope connector, not shown, provided at an end portion of the cable. FIG. 1 shows an electrical connection state in the case.

The endoscope 40 is provided with a signal line VPLIVE for sensing, at the CCU 10, connection of the endoscope connector to the CCU 10. The signal line VPLIVE is, for example, pulled down in the endoscope 40. The analog front end section 20 of the CCU 10 is provided with a signal line VPLIVE, and the signal line VPLIVE is, for example, pulled up on the side of the analog front end section 20, and is connected to the amplifier 26 in the analog front end section 20. The amplifier 26 amplifies a signal appearing in the signal line VPLIVE in the analog front end and supplies the amplified signal to a type determination method discrimination section 22 in the FPGA 20.

When the endoscope connector is connected to the CCU 10, the signal lines VPLIVE in the endoscope 40 and the CCU 10 are connected to each other. Thus, the signal line VPLIVE in the analog front end section 20 is pulled down, for example, so that a voltage level supplied to the amplifier 26 changes from a high level (hereinafter referred to as an H level) to a low level (hereinafter referred to as an L level). The type determination method discrimination section 22 is configured to detect electrical connection of the endoscope connector to the CCU 10 based on a change in output of the amplifier 26.

The endoscope 40 is provided with signal lines CJD1 and CJD2 for performing image pickup device type determination based on the resistive voltage dividing scheme, and the signal lines CJD1 and CJD2 are each connected to a reference potential point via a pull-down resistance not shown. The analog front end section 20 of the CCU 10 is provided with signal lines CJD1 and CJD2. The signal lines CJD1 and CJD2 are each connected to a power supply terminal via a pull-up resistance not shown and are connected to an input end of the A/D conversion section 27.

When the endoscope connector is connected to the CCU 10, the signal lines CJD1 in the endoscope 40 and the CCU 10 are connected to each other, and the signal lines CJD2 are also connected to each other. Thus, a resistive voltage dividing value based on the two resistances connected to the signal line CJD1 is supplied to the A/D conversion section 27. A resistive voltage dividing value based on the two resistances connected to the signal line CJD2 is supplied to the A/D conversion section 27. The A/D conversion section 27 converts the two inputted resistive voltage dividing values into digital signals for output to the type determination method discrimination section 22 in the FPGA 21.

On the other hand, when the endoscope 40 does not support the resistive voltage dividing scheme, the signal lines CJD1 and CJD2 in the endoscope 40 do not exist. In the case, both of the two inputs of the A/D conversion section 27 become, for example, a predetermined H level.

When detecting electrical connection of the endoscope connector to the CCU 10 through the signal line VPLIVE, the type determination method discrimination section 22 outputs the two resistive voltage dividing values from the A/D conversion section 27 to an image pickup device type determination section 23. The image pickup device type determination section 23 is configured to determine the type of the image pickup device mounted on the endoscope 40 based on the two resistive voltage dividing values, and to output the determination result as type determination information to the FPGA 31 in the video processing section 30.

When both of the two outputs from the A/D conversion section 27 keep a value corresponding to the predetermined H level even if a predetermined time period has elapsed after the endoscope 40 is connected to the CCU 10, the type determination method discrimination section 22 is configured to determine that the endoscope 40 does not support the resistive voltage dividing scheme, and to output the determination result as the type determination information to the FPGA 31. That is, in the case, the type determination method discrimination section 22 is configured to determine that the endoscope 40 does not support the resistive voltage dividing scheme and is a next-generation endoscope supporting the communication scheme, and to change a method of type determination from the resistive voltage dividing scheme to the communication scheme.

In the communication scheme, as described above, first it is necessary to supply the endoscope 40 with power and a clock. The analog front end section 20 is provided with the power supply section 28. The power supply section 28 is configured to be able to generate power output having a power supply voltage suitable for the FPGA 41 of the endoscope 40 at the time of type determination of the image pickup device by being controlled by the image pickup device type determination section 23.

A CLK output supplied to each element in the endoscope 40 is obtained by the clock generation section 33 of the video processing section 30. A CXO 34 of the clock generation section 33 outputs a reference oscillation output at a predetermined reference frequency to a PLL section 35 and a frequency division section 32 in the FPGA 31. The PLL section 35 is also given an oscillation output of a VCXO section 36, and outputs to the VCXO section 36 an output for setting a phase difference between a phase of the reference oscillation output and a phase of the oscillation output of the VCXO section 36 to 0. The VCXO section 36 is configured so that an oscillation frequency is controlled by the PLL section 35, and an oscillation output synchronized with the reference oscillation output is obtained from the VCXO section 36. The oscillation output is supplied to the PLL section 35 and a frequency division section 37. The frequency division section 37 frequency-divides the output of the VCXO section 36 to generate a driving clock (CLK) for supply to a selector 24.

The FPGA 31 is given the type determination information indicating the type of the image pickup device from the image pickup device type determination section 23, and is configured to supply a clock control signal for generating a necessary clock to the VCXO section 36 in the clock generation section 33 based on the type determination information. The VCXO section 36 is configured so that a frequency of the oscillation output is controlled according to the clock control signal from the FPGA 31. Thus, based on the clock control signal according to the type determination result of the image pickup device, the oscillation frequency of the driving CLK is controlled to a frequency suitable for driving the image pickup device of the endoscope 40.

The frequency division section 32 in the FPGA 31 can frequency-divide the inputted reference oscillation output to generate a type determining clock (CLK). The FPGA 31 supplies the type determining CLK to the selector 24 of the analog front end section 20. Thus, the type determining CLK and the driving CLK are inputted to the selector 24 from the video processing section 30.

The image pickup device type determination section 23 is configured to stop output of the selector 24 until determining that the endoscope 40 supports the communication scheme, to cause the selector 24 to select the type determining CLK for output when determining that the endoscope 40 supports the communication scheme, and when a determination result of the type of the endoscope 40 is obtained, to then cause the selector 24 to select the driving CLK for output. The type determining CLK from the selector 24 is supplied to the FPGA 41 of the endoscope 40.

Note that although FIG. 1 shows only a power supply line for supplying only the FPGA 41 with power output from the power supply section 28, a power supply line for supplying power output to each element in the endoscope 40 is actually provided from the power supply section 28. Similarly, although FIG. 1 shows only a clock line for supplying only the FPGA 41 with a clock (CLK) output from the selector 24, a clock line for supplying a CLK output to each element in the endoscope 40 is actually provided from the selector 24.

Thus, at the time of type determination of the image pickup device, the endoscope 40 are supplied with the type determining power output and the type determining CLK which are power and CLK outputs different in normal driving including startup of the endoscope 40. The FPGA 41 of the endoscope 40 includes a communication circuit and a memory not shown, and the memory stores image pickup device type information indicating the type of the image pickup device provided in the endoscope 40. The FPGA 41 is supplied with power output and CLK output to start operation. The type determining power output and the type determining CLK in type determination correspond to the FPGA 41, and the FPGA 41 is supplied with power and a clock to start operation normally.

The FPGA 41 is configured to communicate with the communication section 29 of the CCU 10 through the communication circuit at startup to transmit the image pickup device type information to the communication section 29. The communication section 29 enables exchange of information between the FPGA 41 and the FPGA 30. The communication section 29 is configured to supply the image pickup device type information to the FPGA 21 when receiving the image pickup device type information from the FPGA 41. An image pickup communication reception section 25 of the FPGA 21 receives the image pickup device type information from the communication section 29 for output to the image pickup device type determination section 23.

Note that the FPGA 41 of the endoscope 40 is configured to perform image pickup device communication for determining the type of the image pickup device at startup, and after the type determination, to perform scope communication for transmitting scope-specific information including a white scratch and a scope type. The scope-specific information transmitted from the FPGA 41 is supplied by the communication section 29 to the FPGA 31.

When the image pickup device type information is inputted through the image pickup communication reception section 25, the image pickup device type determination section 23 determines the type of the image pickup device based on the inputted image pickup device type information to obtain a determination result. The image pickup device type determination section 23 is configured to output the type determination result to the FPGA 31, and to control the selector 24 and the power supply section 28 based on the determination result. The clock generation section 33 has generated a driving CLK based on the type determination result of the image pickup device. After the image pickup device type determination, the selector 24 supplies the driving CLK as a CLK output to the endoscope 40. The power supply section 28 generates power in a sequence corresponding to the type of the image pickup device for supply to each element the endoscope 40 as power output.

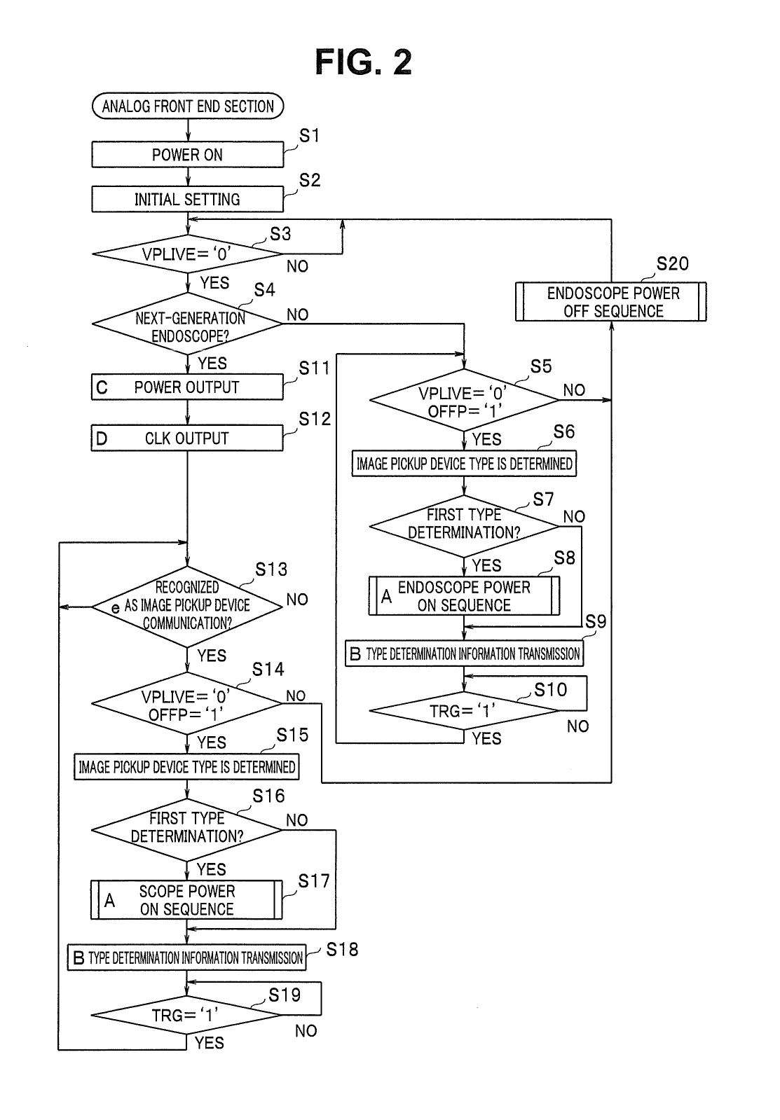

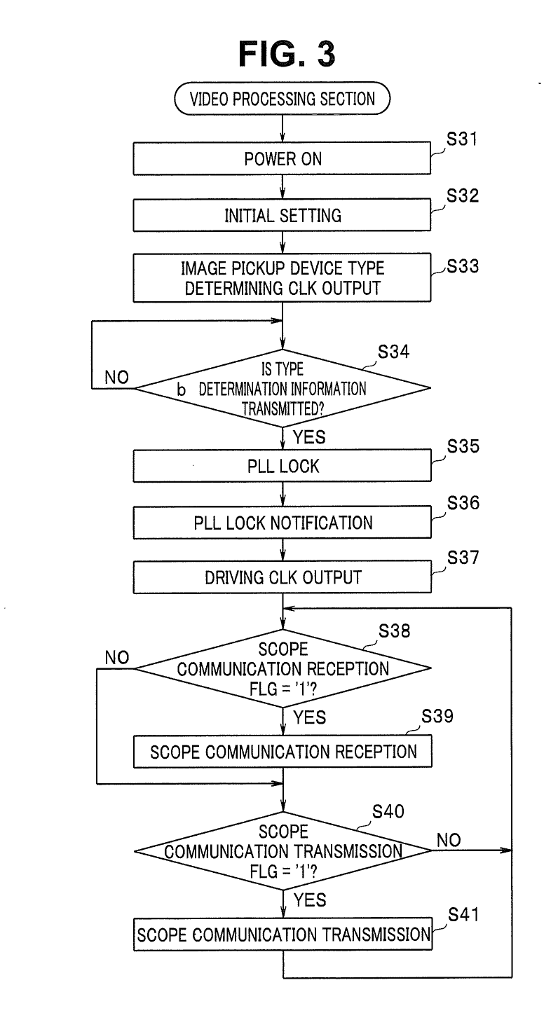

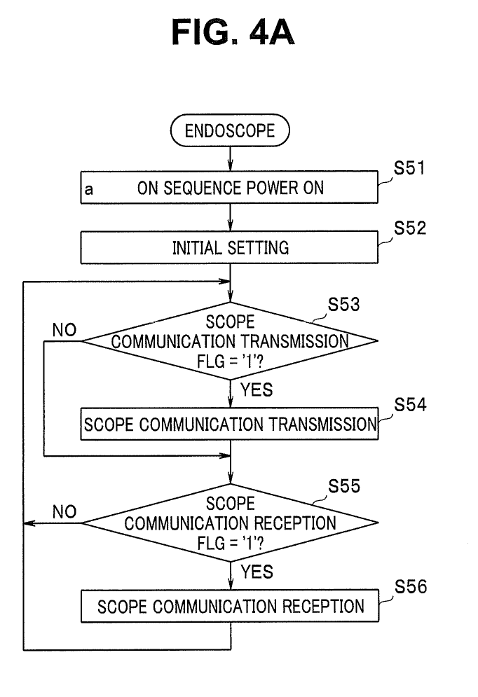

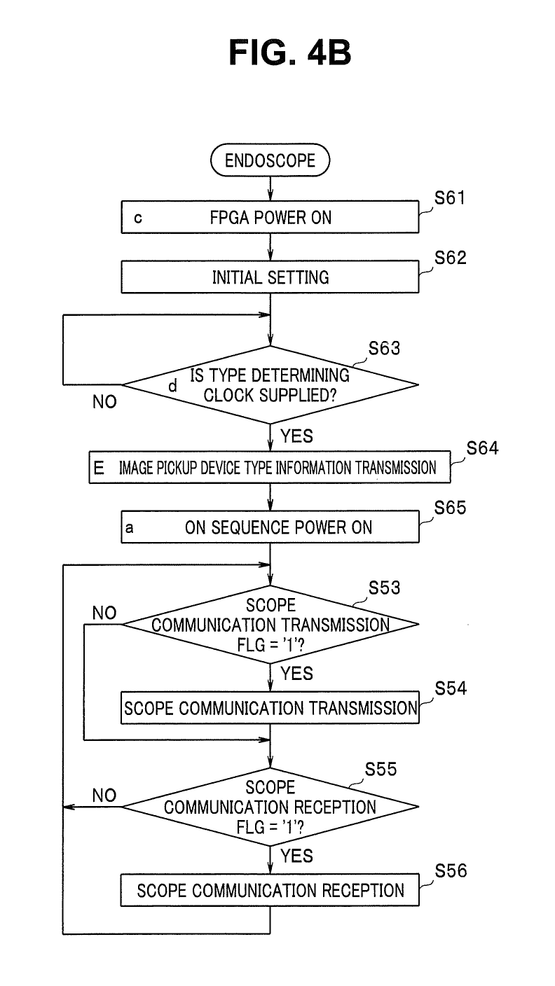

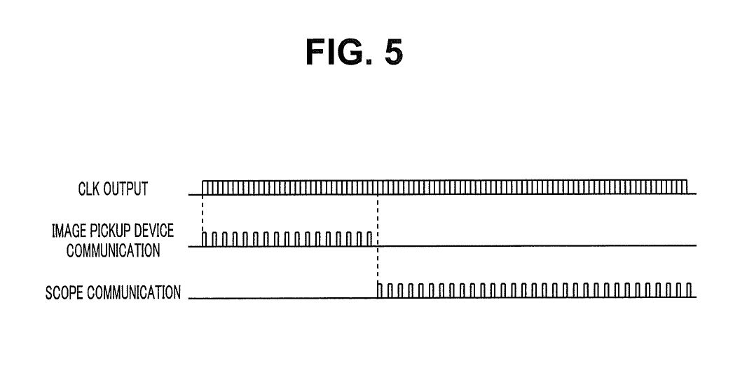

Next, operation of the thus-configured embodiment will be described with reference to FIGS. 2-5. FIG. 2 is a flowchart showing processes in the analog front end section 20, FIG. 3 is a flowchart showing processes in the video processing section 30, and FIGS. 4A and 4B are flowcharts showing processes in the endoscope 40. Note that a-e in respective processing steps in FIGS. 2, 3, 4A and 4B show processes linked to respective processes A-E. FIG. 5 is a timing chart for explaining timing of CLK output, image pickup device communication, and scope communication.

When the SW power supply 38 is turned on, both the analog front end section 20 and the video processing section 30 of the CCU 10 are supplied with power. When being powered on in step S31 in FIG. 3, the video processing section 30 makes a predetermined initial setting in step S32. Note that due to power-on, the video processing section 30 is supplied with an OFFP signal having a logical value of ‘1’ (H level) indicating power-on, and the video processing section 30 gives the OFFP signal to the FPGA 21 of the analog front end section 20. The clock generation section 33 of the video processing section 30 is controlled by the FPGA 31 to generate a clock. The frequency division section 32 of the FPGA 31 frequency-divides the clock from the clock generation section 33 to generate a type determining CLK. The clock from the clock generation section 33 is supplied to the FPGA 21 of the analog front end section 20, and the type determining CLK is supplied to the selector 24 of the analog front end section 20.

The FPGA 21 of the analog front end section 20 is powered on in step S1 in FIG. 2, becomes operable by a clock being inputted from the clock generation section 33, and makes a predetermined initial setting (step S2). The type determination method discrimination section 22 of the FPGA 21 determines whether an output VPLIVE of the amplifier 26 has a logical value of ‘0’ (H level) or not, that is, whether the endoscope 40 is connected to the CCU 10 or not (step S3).

When the endoscope 40 is connected to the CCU 10 so that the output VPLIVE becomes ‘0’, the type determination method discrimination section 22 determines in the next step S4 whether the endoscope 40 is a next-generation endoscope not supporting the resistive voltage dividing scheme or not, that is, whether both of two inputs of the A/D conversion section 27 have been a predetermined H level or not. When neither of the two inputs of the A/D conversion section 27 has been the predetermined H level, the type determination method discrimination section 22 determines that a next-generation endoscope is not connected, and shifts the processing to step S5 to perform type determination based on the resistive voltage dividing scheme.

In step S5, the FPGA 21 determines whether VPLIVE is a logical value of ‘0’ or OFFP is a logical value of ‘1’. When VPLIVE is a logical value of ‘1’, that is, the endoscope 40 is not connected and is not powered on, the processing is shifted to step S20 to execute an OFF sequence for turning off the endoscope power supply. When the endoscope 40 is connected or power is on, the image pickup device type determination section 23 performs type determination of the image pickup device mounted on the endoscope 40 based on the two inputs from the A/D conversion section 27 in step S6.

Note that the FPGA 21 operates based on a clock from the clock generation section 33, and is configured to repeatedly execute type determination based on the resistive voltage dividing scheme for a predetermined time period at timing when a trigger TRG synchronized with the clock becomes ‘1’. When detecting a trigger TRG of ‘1’ in step S10, the FPGA 21 repeatedly executes the processes in steps S5-S9. In step S7, the FPGA 21 determines whether the type determination in step S6 is first determination or not. In the case of first type determination, the FPGA 21 executes a predetermined ON sequence for supplying power to the endoscope based on the type determination result of the image pickup device in step S8. The image pickup device type determination section 23 of the FPGA 21 outputs the type determination result of the image pickup device based on the two resistive voltage dividing values as the type determination information to the FPGA 31 of the video processing section 30 (step S9).

When the type determination information is inputted from the FPGA 21, the FPGA 31 shifts the processing from step S34 to step S35. The FPGA 21 controls the clock generation section 33 based on the type determination information to lock the PLL (step S35). When the PLL is locked in the clock generation section 33, the FPGA 31 transmits a PLL lock notification to the FPGA 21 (step S36), and outputs a driving CLK suitable for driving the image pickup device to the FPGA 31 (step S37). The driving clock CLK is selected by the selector 24 of the FPGA 21 to be supplied to the endoscope 40.

Now, it is assumed that as the endoscope 40, an endoscope adopting the resistive voltage dividing scheme for image pickup device type determination is connected to the CCU 10. In the case, an operation flow shown in FIG. 4A is adopted. The endoscope 40 is powered on through the ON sequence in step S51 in FIG. 4A to start operation. After making a predetermined initial setting in step S52, the endoscope 40 sets a transmission flag FLG of scope communication to ‘1’ in step S53 to perform transmission of scope communication (step S54). That is, the FPGA 41 transmits the scope-specific information to the analog front end section 20.

The communication section 29 of the analog front end section 20 receives the scope-specific information for transfer to the FPGA 31 of the video processing section 30. The FPGA 31 sets a reception flag FLG of scope communication to ‘1’ in step S38 to receive the scope-specific information (step S39). In step S40, when having data to be transmitted to the endoscope 40, the FPGA 31 sets the transmission flag FLG of scope communication to ‘1’ to perform transmission of scope communication (step S41).

The communication section 29 of the analog front end section 20 relays transmitted information of scope communication from the FPGA 31 for transmission to the endoscope 40. The FPGA 41 of the endoscope 40 sets the reception flag FLG of scope communication to ‘1’ in step S55 to receive the transmitted information from the video processing section 30 (step S56).

Next, it is assumed that as the endoscope 40, an endoscope adopting the communication scheme for image pickup device type determination is connected to the CCU 10. In the case, an operation flow shown in FIG. 4B is adopted. When determining in step S4 that a next-generation endoscope adopting the communication scheme is connected, the analog front end section 20 performs power output in step S11. The image pickup device type determination section 23 controls the power supply section 28 to generate power output to be supplied to the FPGA 41 of the endoscope 40.

When the FPGA 41 is powered on in step S61 in FIG. 4B, the endoscope 40 makes a predetermined initial setting in step S62. In step S12, the image pickup device type determination section 23 of the analog front end section 20 controls the selector 24 to output the type determining CLK to the FPGA 41 of the endoscope 40. When being supplied with the type determining CLK in step S63, the FPGA 41 of the endoscope 40 performs image pickup device communication to read and transmit the image pickup device type information stored in the memory (step S64).

FIG. 5 shows the type determining CLK through CLK output, and shows through image pickup device communication that image pickup device communication is repeatedly performed for a predetermined time period at timing when the trigger TRG synchronized with the type determining CLK becomes ‘1’. Each pulse in FIG. 5 shows the trigger TRG, and the trigger TRG causes image pickup device communication to be repeatedly performed, so that transmission of the image pickup device type information is repeated. The image pickup device type information from the endoscope 40 is received in the communication section 29 of the analog front end section 20, and is transferred to the FPGA 21.

The image pickup communication reception section 25 of the FPGA 21 supplies the received image pickup device type information to the image pickup device type determination section 23. When determining in step S13 that image pickup device communication has been performed, the image pickup device type determination section 23 performs type determination of the image pickup device based on the communication scheme in steps S14-S19.

That is, in step S14, the FPGA 21 determines whether VPLIVE is a logical value of ‘0’ or OFFP is a logical value of ‘1’. When VPLIVE is a logical value of ‘1’, that is, the endoscope 40 is not connected and is not powered on, the processing is shifted to step S20 to execute the OFF sequence for turning off the endoscope power supply. When the endoscope 40 is connected or power is on, the image pickup device type determination section 23 performs type determination of the image pickup device mounted on the endoscope 40 based on the image pickup device type information in step S15.

In step S16, the FPGA 21 determines whether the type determination based on the communication scheme in step S15 is first determination or not. In the case of first type determination, in step S17, the FPGA 21 executes the predetermined ON sequence for supplying power to the endoscope based on the type determination result of the image pickup device. The image pickup device type determination section 23 of the FPGA 21 outputs the type determination result of the image pickup device based on the image pickup device type information as the type determination information to the FPGA 31 of the video processing section 30 (step S18). When detecting the trigger TRG of ‘1’ in step S19, the FPGA 21 repeatedly executes the processes in steps S13-S18.

Other actions are similar to the case of the resistive voltage dividing scheme.

Thus, in the embodiment, whether an endoscope to be connected adopts the resistive voltage dividing scheme or the communication scheme as a determination method for the image pickup device, it is possible to reliably determine the type of the image pickup device. Since type determination based on the resistive voltage dividing scheme is performed before type determination based on the communication scheme requiring power-on, it is possible to perform type determination without subjecting an endoscope requiring power-on in a predetermined sequence to power-on ignoring the sequence.

When supply of power output from SW power supply 38 is stopped, the video processing section 30 receives the OFFP signal indicating the stop of power supply at the same time as the stop of power supply. In the case, the video processing section 30 may be configured to output a black image to a monitor which outputs a video signal such as an endoscope image to display black. Thus, it is possible to prevent an unnecessary image from being displayed on the monitor when power is off.

The CCU confirms connection and disconnection of the endoscope when power is on, and when the endoscope is disconnected, the video processing section records a disconnection log. When a reset process (power-off process) is performed in the video processing section when power is on, disconnection information indicating that the endoscope is disconnected is needed even when the endoscope is connected. So, when given a reset process instruction, the analog front end section outputs, to the video processing section, the OFFP signal indicating the reset process to the video processing section and the disconnection information. Thus, reset can be performed in the CCU. However, when the above approach is used, the video processing section has a problem that the disconnection log is recorded due to the disconnection information generated by the reset process even when the endoscope is connected.

So, when the OFFP signal is generated, the FPGA of the video processing section may perform the reset process based on the disconnection information, while controlling recording of the disconnection log to be stopped. Thus, even when power is unexpectedly turned off while the endoscope is connected, it is possible to prevent the disconnection log from being left as a record.

The present invention is not limited to the above each embodiment as it is, and can be embodied by varying components within the scope not deviating from the gist at an implementation stage. And a plurality of components disclosed in the above each embodiment can be appropriately combined to form various inventions. For example, some components of all the components shown in an embodiment may be removed. Furthermore, components over different embodiments may be appropriately combined.

According to the present invention, an object is to provide an endoscope apparatus capable of efficiently performing type determination of an image pickup device even when any of the communication scheme and the resistive voltage dividing scheme is adopted in an endoscope.

The present invention is not limited to the above embodiment, and it is possible to make various changes, variants, or the like within the scope not changing the gist of the present invention.

What is claimed is:

1. An endoscope apparatus comprising:
a first discrimination section configured to be connectable to a first endoscope including a communication circuit and capable of transmitting image pickup device type information, and a second endoscope comprising a resistance configured by a resistance value for enabling identification of a mounted image pickup device and corresponding to the image pickup device, and configured to acquire information for identifying a type of an image pickup device from a connected endoscope to identify a type of the connected endoscope; and
an image pickup communication reception section configured to receive the image pickup device type information from the communication circuit when it is determined in the first discrimination section that the first endoscope is connected.

2. The endoscope apparatus according to claim 1, further comprising
a second discrimination section configured to identify a type of the image pickup device mounted on the first endoscope based on the image pickup device type information.

3. The endoscope apparatus according to claim 1, further comprising:
a power supply section configured to supply the first endoscope with power output for communicating with the first endoscope when it is determined in the first discrimination section that the first endoscope is connected; and
a clock control section configured to supply the first endoscope with a clock for communicating with the first endoscope.

4. The endoscope apparatus according to claim 1, wherein:
the information for identifying the type of the image pickup device mounted on the second endoscope is based on a resistance value of a pull-down resistance provided in the second endoscope; and
the first discrimination section determined that the first endoscope is connected when a voltage based on a voltage drop due to the pull-down resistance includes a value exceeding a predetermined threshold.

5. The endoscope apparatus according to claim 2, wherein:
the information for identifying the type of the image pickup device mounted on the second endoscope is based on a resistance value of a pull-down resistance provided in the second endoscope; and
the second discrimination section converts a voltage based on a voltage drop due to the pull-down resistance into a digital signal to determine the type of the image pickup device mounted on the second endoscope.

6. The endoscope apparatus according to claim 1, wherein
the image pickup communication reception section receives scope-specific information on the first endoscope transmitted from the communication circuit when it is determined in the first discrimination section that the first endoscope is connected.

7. A camera control unit for driving an image pickup device mounted on an endoscope, comprising:
a connection section connectable to a first endoscope comprising a storage area in which image pickup device type information enabling identification of a mounted image pickup device is stored, and a second endoscope comprising a resistance configured by a resistance value for enabling identification of a mounted image pickup device and corresponding to the image pickup device;
a circuit configured to acquire a signal for identifying a type of the image pickup device mounted on the second endoscope through the connection section when the first endoscope or the second endoscope is connected to the connection section; and
a first discrimination section configured to determine whether an endoscope connected to the connection section is the first endoscope or the second endoscope based on the signal acquired by the circuit.

8. The camera control unit according to claim 7, further comprising
an image pickup communication reception section configured to receive the image pickup device type information through a communication circuit when it is determined by the first discrimination section that the first endoscope is connected to the connection section.

9. The camera control unit according to claim 8, further comprising
a second discrimination section configured to identify a type of the image pickup device mounted on the first endoscope based on the image pickup device type information.

10. The camera control unit according to claim 7, further comprising:
a power supply section configured to supply the first endoscope with power output for communicating with the first endoscope when it is determined in the first discrimination section that the first endoscope is connected; and
a clock control section configured to supply the first endoscope with a clock for communicating with the first endoscope.

11. The camera control unit according to claim 10, wherein
the clock control section can supply a type determining clock for identifying a type of an image pickup device and a driving clock for driving the image pickup device as the clock for communicating with the first endoscope.

12. The camera control unit according to claim 11, wherein
the clock control section can switch the clock supplied to the first endoscope between the type determining clock and the driving clock.

13. The camera control unit according to claim 7, wherein:
the information for identifying the type of the image pickup device mounted on the second endoscope is based on a resistance value of a pull-down resistance provided in the second endoscope; and
the first discrimination section determines that the first endoscope is connected when an inputted voltage includes a value exceeding a predetermined threshold.

14. The camera control unit according to claim 9, wherein:
the information for identifying the type of the image pickup device mounted on the second endoscope is based on a resistance value of a pull-down resistance provided in the second endoscope; and
the first discrimination section converts a voltage dividing value based on a pull-up resistance provided in the connection section and the pull-down resistance into a digital signal to determine the type of the image pickup device mounted on the second endoscope.

15. The camera control unit according to claim 8, wherein
the image pickup communication reception section receives scope-specific information on the first endoscope transmitted from the communication circuit when it is determined in the first discrimination section that the first endoscope is connected.
